(19) United States
(12) Patent Application Publication
(10) Pub. No.: US 2016/0175163 A1
(43) Pub. Date: Jun. 23, 2016

(54) WOMENS HYGIENE PAD WITH PLURAL INSERTS

(71) Applicant: Conteena Williams, Discovery Bay, CA (US)

(72) Inventor: Conteena Williams, Discovery Bay, CA (US)

(21) Appl. No.: 14/576,363

(22) Filed: Dec. 19, 2014

(51) Int. Cl. A61F 13/42; A61F 13/84

(52) U.S. Cl. CPC A61F 13/42; A61F 13/8405; A61F 2013/422

(57) ABSTRACT

A women's hygiene pad with plural inserts (or “hygiene pad”) for providing soothing and healing materials, indicators for when to replace, enhanced attachment surfaces, sweat protection, and enhanced odor coverage. The hygiene pad comprises a base pad member, a soothing and healing insert, and a sweat and odor insert of a women's hygiene. The base pad includes a outer moisture resistant layer and an inner moisture absorbent layer, and receives the soothing and healing insert and sweat and odor insert to provide primary leak and odor protection. Along the edges of the base pad are absorbent auxiliary protection strips that provide a supplemental, final barrier for any fluid not absorbed by soothing and healing insert, the sweat and odor insert, or the inner layer of the base pad member.

### BACKGROUND OF THE INVENTION

1. Field of the Invention

This invention relates generally to hygiene accessories and, more particularly, to a wearable women's hygiene insert having moisture protection and notification.

2. Description of the Prior Art

The use and design of conventional sanitary napkins for use by a women while she is menstruating, for lochia, or for any other circumstance where it is necessary to absorb a flow of blood from her vagina is well known. A conventional single use sanitary napkins is customarily worn between the vulva and a woman's undergarments and often include absorbent material to help prevent leaking and wings to add additional leak protection and help secure the pad in place. A problem which still exists, however, is that such conventional sanitary napkins often are unable make a woman feel secure on their insecure days because they may lack soothing and healing materials, indicators for when to replace, enhanced attachment surfaces, sweat protection, and enhanced odor coverage. Thus, there remains a need for an improved women's hygiene pad with plural inserts which would enable a user to selectively include soothing and healing materials to be held in contact with her skin. It would be helpful if such a women's hygiene pad included moisture activated indicators to provide a visual notification of when it should be replaced. It would be additionally desirable for such a women's hygiene pad to be constructed to wick sweat away from the skin and cover odor produced in the area covered.

The Applicant's invention described herein provides for a women's hygiene pad with plural inserts adapted to maximize absorption of bodily fluids. The primary components in Applicant's women's hygiene pad are a base pad, a soothing and healing insert, and sweat and odor insert. When in operation, the women's hygiene pad enables more effective and absorption of fluids and coverage of odors while providing a soothing to a wearer. As a result, many of the limitations imposed by prior art structures are removed.

### SUMMARY OF THE INVENTION

A women's hygiene pad with plural inserts (or “hygiene pad”) for providing soothing and healing materials, indicators for when to replace, enhanced attachment surfaces, sweat protection, and enhanced odor coverage. The hygiene pad comprises a base pad member, a soothing and healing insert, and a sweat and odor insert of a women's hygiene. The base pad includes a outer moisture resistant layer and an inner moisture absorbent layer, and receives the soothing and healing insert and sweat and odor insert to provide primary leak and odor protection. Along the edges of the base pad are absorbent auxiliary protection strips that provide a supplemental, final barrier for any fluid not absorbed by soothing and healing insert, the sweat and odor insert, or the inner layer of the base pad member.

In operation, when the hygiene pad is worn, the soothing and healing insert provides fluid absorbency while dispensing materials with soothing and healing properties, the sweat and odor insert provides fluid absorbency while absorbing odor, and when these inserts become saturated or otherwise no longer function, it causes a moisture indicator to be actuated to remind the wearer to replace the hygiene pad.

It is an object of this invention to provide an improved women's hygiene pad with plural inserts which would enable a user to selectively include soothing and healing materials to be held in contact with her skin.

It is another object of this invention to provide a women's hygiene pad that includes moisture activated indicators to provide a visual notification of when it should be replaced.

It is yet another object of this invention to provide a women's hygiene pad constructed to wick sweat away from the skin and cover odor produced in the area covered.

These and other objects will be apparent to one of skill in the art.

### BRIEF DESCRIPTION OF THE DRAWINGS

FIG. 1 is a top plan view of a soothing and healing insert of a women's hygiene pad built in accordance with the present invention.

FIG. 2 is a top plan view of a sweat and odor insert of a women's hygiene pad built in accordance with the present invention.

FIG. 3 is a top plan view of a women's hygiene pad built in accordance with the present invention.

FIG. 4 is a side cross-sectional view of a women's hygiene pad built in accordance with the present invention

FIG. 5A is a bottom plan view of a women's hygiene pad built in accordance with the present invention saturated with liquid.

FIG. 5B is a bottom plan view of a women's hygiene pad built in accordance with the present invention free of liquid.

FIG. 6 is a top plan view of an embodiment of a women's hygiene pad built in accordance with the present invention having extended wing attachment sections.

### DETAILED DESCRIPTION OF THE INVENTION

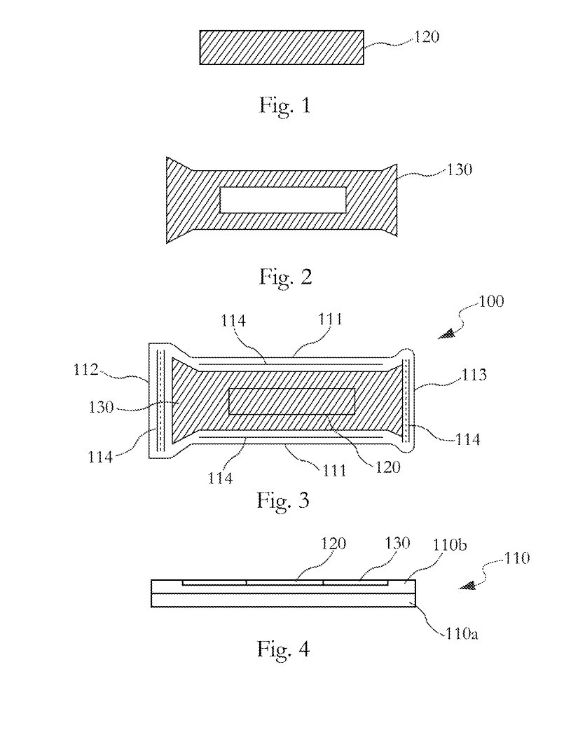

Referring now to the drawings and in particular FIGS. 1, 2, 3, and 4, a women's hygiene pad with plural inserts (or “hygiene pad”) 100 is shown as a base pad member 110, a soothing and healing insert 120, and a sweat and odor insert 130 of a women's hygiene. The base pad 110 is sized, shaped, and otherwise constructed in a substantially similar manner as conventional sanitary napkins, thereby configuring it to be placed between the vulva and a woman's undergarments while in use. Accordingly, the base pad 110 includes a outer moisture resistant layer 110a and an inner moisture absorbent layer 110b. In the preferred embodiment, the base pad 110 includes side wings sections 111, a front section 112 for positioning on the front side of a woman, and a back section 113 for positioning on the back of a woman. Disposed along the side wing sections 111 and across the front section 112 and back section 113 are absorbent auxiliary protection strips 114 that provide a supplemental, final barrier for any fluid not absorbed by soothing and healing insert 120, the sweat and odor insert 130, or the inner layer of the base pad member 110.

The soothing and healing insert 120 attaches to the center of the inner layer 110b of the base pad 110 with an adhesive in place on the surface of the soothing and healing insert 120 facing the base pad 110. The soothing and healing insert 120 defines a permeable strip having small perforations therein and containing conventional absorbent gel members, aloe vera gel, and vitamin E gel. It is contemplated that when a woman is wearing the hygiene pad 100 with the soothing and healing insert 120 in place therein, the gel members will always remain trapped in the strip while when the woman is seated, small amounts of aloe vera gel and vitamin e gel will exit the strip through the perforations and contact the skin of the wearer.

The sweat and odor insert 130 attaches to the inner layer 110b of the base pad 110 surrounding the area where the sweat and odor insert 130 attaches with an adhesive in place on the surface of the soothing and healing insert facing the base pad 110. The sweat and odor insert 130 defines a permeable strip having small perforations therein and containing conventional absorbent gel members. It is contemplated that when a woman is wearing the hygiene pad 100 with the sweat and odor insert 130 in place therein, in addition to merely absorbing fluid not captured by the soothing and healing insert 120 (such as excess blood from the vagina or sweat generated from the area surrounding the vulva), the fluid is absorbed by the gel, thereby mitigating odor emissions.

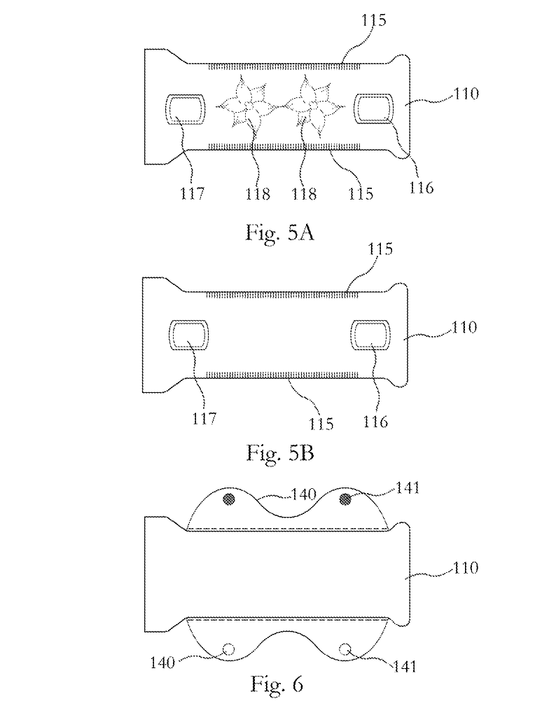

Referring now to FIGS. 3, 4, 5A and 5B, the outer surface of the base pad 110 includes hook fastening surface 115 (from a fabric hook and loop fastener) and two adhesive strips 116 near the front section 112 and back section 113 for adhering to the undergarment of a wearer of the hygiene pad 100. Surrounding the adhesive strips 116 are a fragrance treatment section 117, defined in the preferred embodiment as a cashmere and sandlewood oil treatment, that emits a fragrant aroma from hygiene pad 100.

Moisture indicators 118 are integrated into the material of the base pad 110 and arranged adjacent to one another. The moisture indicators 118 are created with hydrochromic ink, and become invisible when the inner layer 110b of the base pad becomes moist. It is contemplated, however, that due to the inclusion of the soothing and healing insert 120 and sweat and odor insert 130, the inner layer 110b will only begin to get wet once said inserts become saturated. Thus, the moisture indicators 118 are able to provide a visual indication of when it is time to change the hygiene pad 100 because it only indicates when the inserts are saturated or otherwise no longer able to absorb fluid. It is contemplated that the moisture indicators 118 are integrated in the inner layer 110b and the outer layer 110a around them is sufficiently transparent to allow their presence and absence to be observed by a user.

Referring now to FIG. 6, to improve its ability to attach to a woman's undergarments, a hygiene pad 100 may additionally include extended wing members 140 in place of the hook fastening surface 115. It is contemplated that such wing members 140 would desirably include an attachment surface, defined in one embodiment as a hook and loop fastener surface 141, to enhance the removable attachment of the hygiene pad's 100 attachment to a woman's undergarments.

The instant invention has been shown and described herein in what is considered to be the most practical and preferred embodiment. It is recognized, however, that departures may be made therefrom within the scope of the invention and that obvious modifications will occur to a person skilled in the art.

What is claimed is:

1. A women's hygiene pad, comprising:
a base pad configured to be placed between the vulva and a woman's undergarments while in use, wherein said base pad includes an absorbent inner layer and a moisture resistant outer layer; and
a first insert attached to said inner layer, wherein said first insert provides fluid absorbency while dispensing materials with soothing and healing properties on the skin of a wearer of the women's hygiene pad.

2. The women's hygiene pad of claim 1, additionally comprising a second insert attached to said inner layer and adjacent to said first insert, wherein said second insert provides fluid absorbency while absorbing odor and said second insert is arranged to surround the first insert on said inner layer.

3. The woman's hygiene pad of claim 2, wherein said wherein said first insert includes at least one of aloe vera and vitamin E for dispensing on the skin of a wearer of the women's hygiene pad.

4. The women's hygiene pad of claim 1, wherein said base pad includes a plurality of moisture indicators that provide a visual indication when the inner layer becomes moist.

5. The women's hygiene pad of claim 4, wherein said plurality of moisture indicators are disposed adjacent to one another on the base pad.

6. The women's hygiene pad of claim 1, wherein said base pad includes a fragrance treatment section that emits a fragrant aroma therefrom.

7. The women's hygiene pad of claim 1, wherein said base pad includes at least one discrete fastening sections for removably attaching it to a woman's undergarment.

8. The women's hygiene pad of claim 7, wherein said base pad includes three discrete fastening sections, two disposed on opposing sides and one running along the middle of the base pad.

9. The women's hygiene pad of claim 7, wherein at least one discrete fastening sections defines separate fabric hook surfaces running along on opposing sides of the base pad and an adhesive strip running along the middle of the base pad.

10. A women's hygiene pad, comprising:
a base pad configured to be placed between the vulva and a woman's undergarments while in use, wherein said base pad includes an absorbent inner layer and a moisture resistant outer layer; and
a first insert attached to said inner layer, wherein said first insert provides fluid absorbency while absorbing odor.

11. The women's hygiene pad of claim 10, additionally comprising a second insert attached to said inner layer and adjacent to said first insert, wherein said second insert provides fluid absorbency while dispensing materials with soothing and healing properties on the skin of a wearer of the women's hygiene pad

12. The woman's hygiene pad of claim 11, wherein said wherein said second insert includes at least one of aloe vera and vitamin E for dispensing on the skin of a wearer of the women's hygiene pad.

13. The women's hygiene pad of claim 10, wherein said base pad includes a plurality of moisture indicators that provide a visual indication when the inner layer becomes moist.

14. The women's hygiene pad of claim 13, wherein said plurality of moisture indicators are disposed adjacent to one another on the base pad.

15. The women's hygiene pad of claim 10, wherein said base pad includes a fragrance treatment section that emits a fragrant aroma therefrom.

16. The women's hygiene pad of claim 10, wherein said base pad includes at least one discrete fastening sections for removably attaching it to a woman's undergarment.

17. The women's hygiene pad of claim 16, wherein said base pad includes three discrete fastening sections, two disposed on opposing sides and one running along the middle of the base pad.

18. The women's hygiene pad of claim 16, wherein at least one discrete fastening sections defines separate fabric hook surfaces running along on opposing sides of the base pad and an adhesive strip running along the middle of the base pad.
